(19) United States
(12) Patent Application Publication
(10) Pub. No.: US 2002/0106787 A1
(43) Pub. Date: Aug. 8, 2002

(54) Device for repid DNA sample processing with integrated liquid handling, thermocycling, and purification

(76) Inventors: James Benn, Arlington, MA (US); Jean-Francois Manchec, Natick, MA (US)

Correspondence Address: LAHIVE & COCKFIELD, 28 STATE STREET, BOSTON, MA, 02109, US

(21) Appl. No.: 09/939,520

(22) Filed: Aug. 24, 2001

Related U.S. Application Data
(63) Continuation-in-part of application No. 09/561,764, filed on Apr. 28, 2000.
(60) Provisional application No. 60/228,239, filed on Aug. 25, 2000.
(60) Provisional application No. 60/266,035, filed on Feb. 2, 2001.
(60) Provisional application No. 60/131,660, filed on Apr. 29, 1999.
(60) Provisional application No. 60/155,299, filed on Sep. 21, 1999.

(51) Int. Cl. C12M 1/00

(52) U.S. Cl. 435/303.1

(57) ABSTRACT

A flat plate dialysis and ultrafiltration cell and system are provided, having a sample chamber, syringe docking port, with a seal capable of providing a fluid-tight seal after being pierced by a needle, a needle stop capable of preventing a needle from entering the sample chamber, and a needle guide formed in a funnel shape in the syringe docking port to guide a needle toward the sample chamber. The sample chamber is also provided with a dialysis or ultrafiltration membrane provided along a portion of the chamber. The sample chamber is fluidly coupled to a distal end of the syringe docking port and a vent hole.

### REFERENCE TO RELATED APPLICATIONS

This application claims priority to U.S. Provisional Patent Application No. 60/228,239 filed Aug. 25, 2000 and U.S. Provisional Patent Application No. 60/266,035, filed Feb. 2, 2001, the contents of which are hereby incorporated by reference. The subject matter of this application relates to U.S. Provisional Application Nos. 60/131,660, filed Apr. 29, 1999, 60/155,299, filed Sep. 21, 1999, U.S. patent application No. 09/422,677, filed Oct. 21, 1999, U.S. Continuation-in-Part Application No. 09/561,764, filed Apr. 28, 2000 and U.S. Patent Application, Attny. Docket No. GEN-007ACP, filed Aug. 24, 2001. The aforementioned applications, and the references cited therein, are incorporated herein by reference.

### FIELD OF THE INVENTION

The invention relates to devices and methods for high speed, low volume automated sample handling of biological samples, which are useful in the field of genomics for a variety of processes, including DNA sequencing, genetic analysis, and gene expression analysis. The invention further relates to devices and methods for preparing and executing assays for high throughput compound screening for pharmaceutical applications.

### BACKGROUND OF THE INVENTION

Laboratory automation has played a key role in the advancement of genomics and drug discovery over the past decade. Automated systems are now used in high-throughput sample preparation for DNA sequencing at large sequencing centers.

Modern laboratories employ partially automated procedures for handling samples. In these procedures, reagents and templates are combined by manually feeding 96-channel pipettors with thermocycling plates.

The techniques of dialysis and ultrafiltration, although well established, are typically difficult to perform on small sample volumes without suffering loss of the sample. A significant drawback in standard 5-10 μl sequencing reactions is that at least 50% of the sample is wasted. Furthermore, the amount of fluorescently labeled DNA that can be detected on current sequencing machines is much lower than the amounts that are typically processed. Generally, 0.5-1 μl samples are sufficient to detect fluorescently labeled DNA.

### SUMMARY OF THE INVENTION

The present invention addresses the foregoing by providing a flat plate dialysis or ultrafiltration cell having a syringe docking port, a sample chamber fluidly coupled to a distal end of the syringe docking port, and a vent hole fluidly coupled to the sample chamber. The flat plate cell further includes a membrane for separating or filtering a sample by means of unequal diffusion, e.g., by size exclusion.

According to another aspect, the present invention provides a flat plate dialysis system, comprising a needle guide having an upper surface and a bottom surface, the upper surface being adapted to receive a needle, an upper channel plate mounted along an upper surface to the bottom surface of the needle guide, and at least one dialysis chamber formed along a bottom surface of the upper channel plate.

According to another aspect, the present invention involves a method for performing dialysis on a biological sample comprising the steps of providing a dialysis chamber having a flat dialysis membrane along one side of the dialysis chamber, a syringe docking port, and a vent hole each located near an opposite end of the dialysis chamber. The method also includes the steps of injecting a sample from a needle through a syringe docking port into a dialysis chamber and into contact with a first side of the dialysis membrane, and applying a dialysis solution to a second side of the dialysis membrane opposite the first side of the dialysis membrane.

According to another aspect, a flat plate ultrafiltration system is provided. The flat plate ultrafiltration system comprises a syringe docking port, a sample chamber fluidly coupled to a distal end of the syringe docking port, an ultrafiltration membrane provided along a portion of the sample chamber and a device for applying a pressure differential to a sample in the sample chamber.

According to another aspect, a method for performing ultrafiltration on a biological sample is provided. The method comprises providing a sample chamber having an ultrafiltration membrane a syringe docking port in fluid communication with said sample chamber, injecting a sample from a needle through the syringe docking port into the sample chamber and into contact with a first side the ultrafiltration membrane, and applying a pressure differential to the sample chamber to perform ultrafiltration of the sample.

According to another aspect a method for detecting the presence or absence of a first nucleotide, at a position within a strand of DNA in a sample using the flat plate system is provided.

According to another aspect, a method of performing a polymerase chain reaction is provided. The method comprises providing a sample chamber having a membrane along one side of the sample chamber and having a syringe docking port in fluid communication with the sample chamber, wherein the membrane incorporates appropriate reagents for the performance of a polymerase chain reaction. The method further comprises injecting a biological sample from a needle through the syringe docking port into said sample chamber and into contact with a first side of said membrane and imposing conditions on the sample chamber such that a polymerase chain reaction occurs.

According to another aspect, a packaged kit for performing a polymerase chain reaction, comprising a flat plate separation system comprising a sample chamber having a membrane along one side of the sample chamber and having a syringe docking port in fluid communication with the sample chamber, wherein the membrane is impregnated with nucleotide probes and dried, the flat plate separation system packaged with instructions for performing a polymerase chain reaction, is provided.

According to a final aspect, a syringe docking port is provided having a needle stop capable of stopping a needle, a seal mounted against the needle stop and capable of providing a fluid-tight seal after being pierced by the needle and a needle guide mounted against the seal to guide the needle through the seal and toward the needle stop.

### BRIEF DESCRIPTION OF THE DRAWINGS

The foregoing and other objects, features and advantages of the invention will be apparent from the following description, and from the accompanying drawings, in which like reference characters refer to the same parts throughout the different views. The drawings illustrate principles of the invention and, although not to scale, may if necessary show relative dimensions.

FIG. 1 is a cross-sectional view of one embodiment of the invention, illustrated with a syringe needle docking system;

FIG. 2 is a bottom view of a dialysis chamber of the embodiment of FIG. 1;

FIG. 3 is an exploded perspective view of a second embodiment of the invention;

FIG. 4 is a view of a top surface of a needle guide of the second embodiment of the invention;

FIG. 5 is a cross-sectional view of a portion of the needle guide illustrated in FIG. 4;

FIG. 6 illustrates a bottom surface of the needle guide illustrated in FIG. 4;

FIG. 7 shows a top surface of an upper channel plate according to the second embodiment of the invention;

FIG. 8 shows a cross-sectional view of a portion of the upper channel plate illustrated in FIG. 7;

FIG. 9 provides a bottom surface view of the upper channel plate illustrated in FIG. 7;

FIG. 10 is an upper surface view of a lower channel plate according to the second embodiment of the invention;

FIG. 11 is a detailed view of a portion of the upper surface of the lower channel plate illustrated in FIG. 10;

FIG. 12 provides a bottom surface view of the lower channel plate illustrated in FIG. 10 according to the second embodiment of the invention;

FIG. 13 provides an upper surface view of a manifold according to the second embodiment of the invention;

FIG. 14 provides a bottom surface view of the manifold illustrated in FIG. 13; and

FIG. 15 is a cross-sectional view of an alternate embodiment of the invention, wherein the flat plate system is used for ultrafiltration of a sample.

FIG. 16 provides dialysis results of an example of an embodiment of the present invention.

### DETAILED DESCRIPTION OF THE INVENTION

Before further description of the invention, certain terms employed in the specification, examples and appended claims are, for convenience, defined below.

The term “biological sample” refers to a sample comprising one or more cellular or extracellular components of a biological organism. Such components include, but are not limited to, nucleotides (e.g., DNA, RNA, fragments thereof and plasmids), peptides (e.g., structural proteins and fragments thereof, enzymes, etc.), and carbohydrates, etc. The biological samples described herein may also include transport media, biological buffers and other reagents well known in the art for carrying out the processes described above. Although the methods of the invention can be carried out with a biological sample of just about any volume, biological samples in accordance with the invention typically have microliter (μL) volumes and therefore can be referred to as microsamples, e.g., biological microsamples. The methods of the invention are advantageously practiced with biological samples having volumes ranging between about 10 μl and about 0.05 μL, and preferably between about 0.1 μL and about 3 μL.

The term “dialysis” is art-recognized and is understood to refer to the separation or filtering of substances in solution by means of their unequal diffusion through a membrane, including the following forms of dialysis. As used herein, “equilibrium dialysis” refers to dialysis which occurs without exchange or flow of dialysate, e.g. dialysis solution. “Flow dialysis” refers to dialysis which occurs with a flow (or counterflow) of dialysate. “Exchange dialysis” refers to dialysis which includes at least one change of the dialysate surrounding the membrane.

The term “membrane” as used herein refers to both dialysis membranes and ultrafiltration membranes, as appropriate, to accomplish dialysis or ultrafiltration. The membrane is a material of any suitable composition and size which may used to separate or filter substances in solution by means of unequal diffusion, e.g., by size exclusion. Although dialysis membranes and ultrafiltration membranes typically are semipermeable, the term “membrane” as used herein is not so limited. Dialysis membranes and ultrafiltration membranes are closely related and are interchangeable as used herein. In most applications, ultrafiltration membranes are generally designed to withstand elevated pressures.

The term “purification” is intended to encompass, in its various grammatical forms and synonyms (e.g., purification, purifying, clean up, etc.) any operation whereby an undesired component(s) is/are separated or filtered from a desired component(s). Such operations include, but are not limited to, filtration, ultrafiltration, dialysis/equilibrium dialysis, chromatography, and the like. In certain embodiments, purification is achieved by molecular size discrimination among the components of the biological sample. Purification by molecular size discrimination can be achieved using any number of materials of varying porosity well known in the art including, but not limited to, filters, membranes, and semipermeable ultrafiltration filter materials.

The terms “sample chamber”, “dialysis chamber” and “ultrafiltration chamber” are used interchangeably to refer to a chamber suitable for holding a sample to be processed. The term “dialysis chamber” refers to a chamber used to hold a sample for performing dialysis and the term “ultrafiltration chamber” refers to a chamber used to hold a sample for performing ultrafiltration.

The terms “temperature processing,” “temperature treating,” and “thermal processing” are used interchangeably herein to refer to the application of a variety of temperature conditions to the sample, depending on the particular process underway and include, but are not limited to, continuous and discontinuous heating regimens, e.g., denaturation, annealing, incubation, precipitation, and the like. For example, the terms broadly encompass thermocycling associated with PCR and similar processes.

The term “ultrafiltration” refers to any method of purification, separation or filtration wherein the sample is under positive or negative pressure.

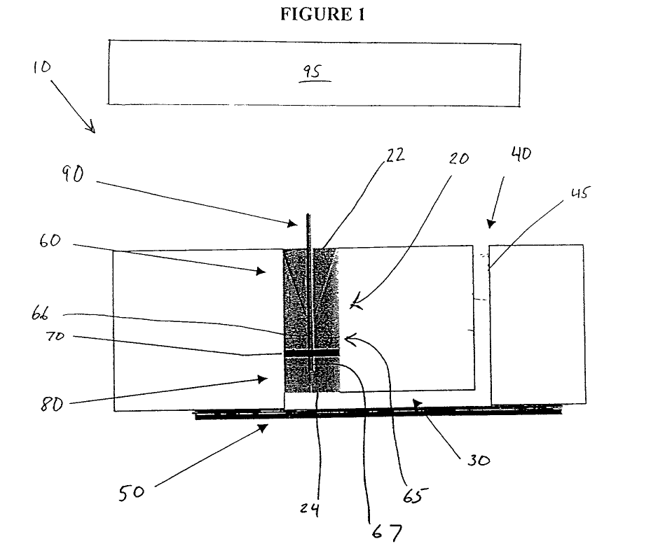

According to a first embodiment of the invention, a flat plate dialysis cell 10 is provided as shown in FIG. 1. The flat plate dialysis cell 10 includes a syringe docking port 20 fluidly coupled with a sample chamber 30 and a vent hole 40. A membrane 50 is provided along a portion of the sample chamber 30.

The syringe docking port 20 preferably includes a needle guide 60, a seal 70 and a needle stop 80. Those of ordinary skill will recognize that the docking port 20 can comprise greater or fewer components, and can have any suitable size and shape. The syringe docking port 20 includes an entry portion 22 opposite a distal end 24. The optional needle guide 60 defines an insertion axis 90, which is preferably perpendicular to the membrane 50. The needle guide 60, formed near entry portion 22, is preferably funnel shaped so as to guide a syringe along a path intersecting the insertion axis 90. The needle guide 60 is preferably formed of polyethylene. However, other non-reactive materials may be used to form the needle guide 60.

The optional seal 70 is preferably formed to provide a fluid-tight seal within the syringe docking port 20. The seal 70 is designed to be repeatedly pierced by a syringe while maintaining the ability to provide a fluid-tight seal. The seal 70 is preferably pierced upon manufacture. Alternatively, the seal 70 may be manufactured without piercing and later pierced by a sharp needle during use. The seal 70 may be formed of silicone, rubber, silicone rubber, or other elastic material, although silicone rubber is preferred.

The optional needle stop 80 can be formed near the distal end 24 of the syringe docking port 20 to prevent a needle from piercing the membrane 50, preferably by preventing the needle from entering the sample chamber 30. As with the needle guide 60, the needle stop 80 is formed of a non-reactive material, such as polyethylene.

The flat plate dialysis cell 10 includes the sample chamber 30. The sample chamber 30 is preferably formed with the distal end 24 of the syringe docking port 20 fluidly coupled to one end of the sample chamber 30. A vent hole 40 is also fluidly coupled to the sample chamber 30, preferably near an opposite end of the sample chamber 30. An optional seal 45 or valve may be provided in or in fluid communication with vent hole 40 to provide for the control of pressure within the sample chamber 30. The membrane 50 is preferably provided along a portion of the sample chamber 30.

Although a primary use of the invention involves dialysis, alternate membranes may be provided within the scope of the invention to provide additional functionality. By way of example, a plastic barrier may be mounted to the bottom surface of the upper channel plate to allow thermocycling of the sample chamber. An optional heat exchanger may also be mounted to the bottom of the plastic barrier. According to an alternate embodiment, the membrane 50 of the flat plate dialysis cell 10 may incorporate enzymes and other reagents necessary for performing PCR (polymerase chain reaction), to allow use of the flat plate dialysis cell 10 in performing in situ PCR. A wide variety of other membranes may be provided to conduct additional processes. In one variation of the invention, a membrane is formed to provide a molecular weight cut-off about 100 Kdal.

The flat plate dialysis cell 10 of the invention may be used to purify biologic samples less than one microliter by the use of the membrane 50. The membrane 50 may be used to retain molecules of interest and allow unwanted molecules to pass through the membrane, out of the sample, by means of dialysis.

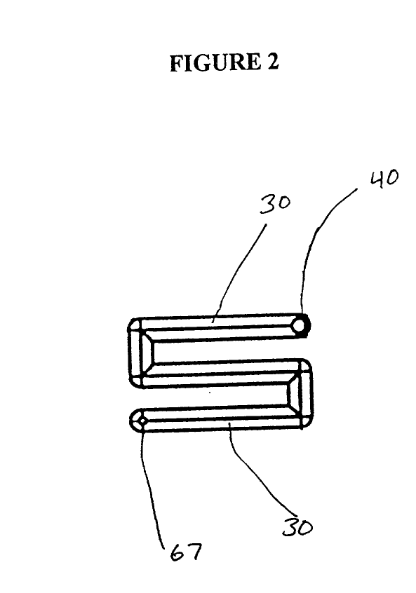

The sample chamber 30, shown in FIG. 2, is preferably formed with a diameter of less than 1 mm and greater than 0.1 mm, preferably having a volume of less than 1 microliter. A diameter of approximately 0.5 is preferred. The sample chamber is preferably formed in the shape of an elongated tube cut along its longitudinal axis, thereby forming a flat portion along substantially all its length. The sample chamber may be formed in a serpentine shape, such as an S shape as is shown in FIG. 2, or may be straight. The sample chamber 30 shown in FIG. 2 shows a lower portion 67 of the guide channel 65 in fluid communication with the sample chamber 30, near an end of the sample chamber 30. A vent hole 40 is also illustrated in fluid communication near an opposite end of the sample chamber 30.

In operation, a needle containing a sample is introduced into the syringe docking port 20. The needle guide 60 guides the needle onto insertion axis 90 and into seal 70. The needle stop 80 prevents the needle from being inserted too far. The needle introduces the sample into the sample chamber 30 preferably through needle stop 80. The vent hole 40 allows for the escape of air from the sample chamber 30 as the sample is introduced. To effect dialysis, the portion of the sample chamber 30 having the membrane 50 is exposed to a dialysis solution. The dialysis solution allows smaller molecules to pass through the membrane 50 from the sample out of the sample chamber 30. Upon completion of the dialysis, the needle previously used to insert the sample, or a different needle, removes the sample from the sample chamber through needle stop 80. The needle may optionally be removed from syringe docking port 20 during dialysis and reinserted upon completion of dialysis to effect removal of the sample.

Optional washing of the flat plate dialysis cell 10, or any part thereof, may then be performed. Preferably, an alcohol-based solution is used. Washing may be performed with or without disassembly of the flat plate dialysis cell 10.

The flat plate dialysis cell 10 is easily optionally multiplied into an array of multiple flat plate dialysis cells, allowing each flat plate dialysis cell 10 to use a portion of a single, continuous dialysis membrane 50.

The invention is capable of processing many samples in parallel, if desired, using standard micro-titer plates as reagent sources. The system can be used to retrieve, mix and dispense fluids by integration with air or liquid-filled volumetric devices, such as piezoelectric elements, movable pistons or syringe-type plungers.

A syringe needle docking system 95, shown in FIG. 1, may be used to automate the insertion and removal of samples. The syringe needle docking system 95 may optionally include automated syringe needle movement and automated syringe plunger actuation.

The dimensions of the sample chamber 30 provide for the use of small sample volumes while providing a large surface area for the sample to be in contact with the membrane 50. It is desirable to maximize the surface area of the sample chamber 30 along the membrane 50 for a given sample chamber 30 volume. However, the surface tension of the sample is an important consideration to allow for the maximum recovery of a sample from the sample chamber by a needle through the needle stop 80. Preferably, the sample chamber 30 diameter is between 1.0 mm and 0.1 mm. Specifically, approximately 0.5 mm is preferred. A large surface area along the membrane 50 allows for more rapid dialysis of a sample. This large surface area is provided without need for additional components, such as those disclosed in U.S. Pat. No. 5,679,310 to Manns.

In one variation of the invention, thermocycling can be performed involving a hot and/or cold air or liquid to change the sample temperature. Simple air blowers or blowing ambient air and air heated by resistance heaters over the sample chamber 30 may be used to change the temperature. In one variation, a plastic membrane is used along a portion of sample chamber 30. A temperature controlled gas, such as air, or fluid is then passed along the plastic membrane to control the temperature of the sample chamber 30 and a sample therein. The temperature may be measured and controlled by standard controllers. The heating rate may be increased as desired by using, for example, superheated air for the first part of the heating cycle, then cooler air to avoid excessive overshoot of the temperature of the sample chamber 30. The present invention is well suited to thermocycling of submicroliter samples, as the temperature of each of the sample chambers 30 can quickly and easily be controlled as described above.

The present invention provides for effective removal of contaminants from a thermocycling reaction. Once the reaction mixture is thermocycled, purification may be achieved by placing the mixture into the sample chamber 30 with the membrane 50, which is in contact with a dialysis solution having a lower concentration of ionic components. The difference in osmotic pressure across the membrane 50 forces contaminants in the product to migrate across the membrane 50 into the dialysis solution, effectively removing them from the product.

In another variation of the invention, in situ PCR can be performed using the flat plate dialysis cell of the illustrative embodiment. To perform in situ PCR, the membrane 50 of the flat plate dialysis cell 10 may further be impregnated (on the sample chamber side) with nucleotide probes. As used herein, the term “nucleotide probes” refers to suitable reagents for performing a polymerase chain reaction, including the labeled primers used in the SNP (single nucleotide polymorphism) assay described in U.S. Pat. No. 5,391,480, the contents of which are incorporated herein by reference, oligonucleotide primers, labeled or unlabeled nucleotides, and labeled or unlabeled dideoxynucleotides. These probes can generally be dried down on the membrane 50 and stored for months or more on the membrane surface. The membrane 50 is then covered with a thin water-impermeable membrane, such as a mylar membrane, to allow eventual thermocycling of the sample chamber 30 without loss of water due to evaporation through the membrane. In preparation for thermocycling, the target DNA sample is loaded into the sample chamber 30 and conditions for performing a polymerase chain reaction are imposed such that a polymerase chain reaction occurs. As used herein, “conditions” refers to the addition of enzymes, e.g. a proofreading polymerase, magnesium ions, heat and other materials, for the performance of a polymerase chain reaction, as described in, for example, U.S. Pat. No. 5,391,480 and U.S. Pat. No. 4,683,195, the contents of which are incorporated herein by reference.

After the PCR reaction is complete, the thin mylar membrane is peeled off or removed from the surface of the membrane 50, and a dialysis or ultrafiltration protocol as described herein is performed, such that the primers or unincorporated tagged nucleotides are removed from the PCR reaction, providing for further processing of the DNA sample or for detection of the presence or absence of a particular nucleotide in the DNA sample.

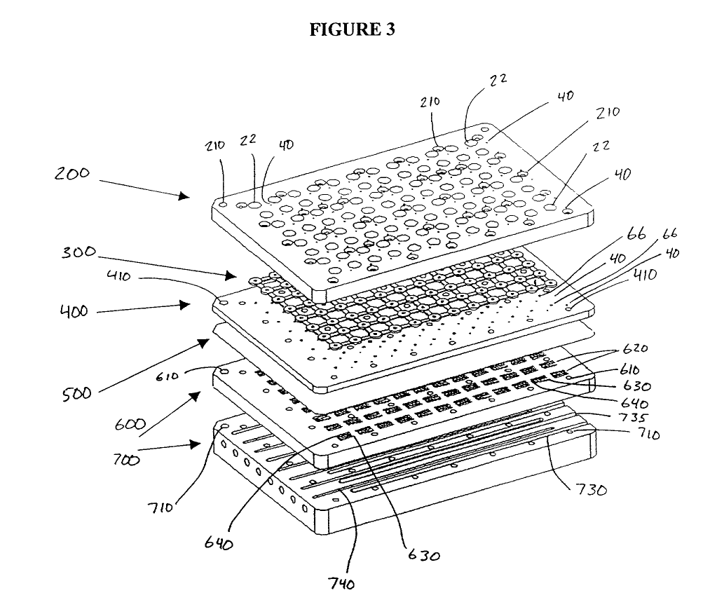

Another embodiment of the invention is shown in FIG. 3. The flat plate dialysis system 100 shown in FIG. 3 preferably includes a needle guide 200, a seal 300, an upper channel plate 400, a membrane 500, a lower channel plate 600 and a manifold 700. Preferably, a plurality, such as 96 or 384 or more, flat plate dialysis cells 10 are provided in the flat plate dialysis system 100, as described below. Those of ordinary skill will recognize that any suitable number of cells can be employed.

A needle guide 200 is provided with a plurality of holes. For each flat plate dialysis cell 10, an entry portion 22 and a vent hole 40 are preferably provided within needle guide 200. Alignment holes 210 are also preferably provided to aid in mounting of the various components of the flat plate dialysis system 100 to each other.

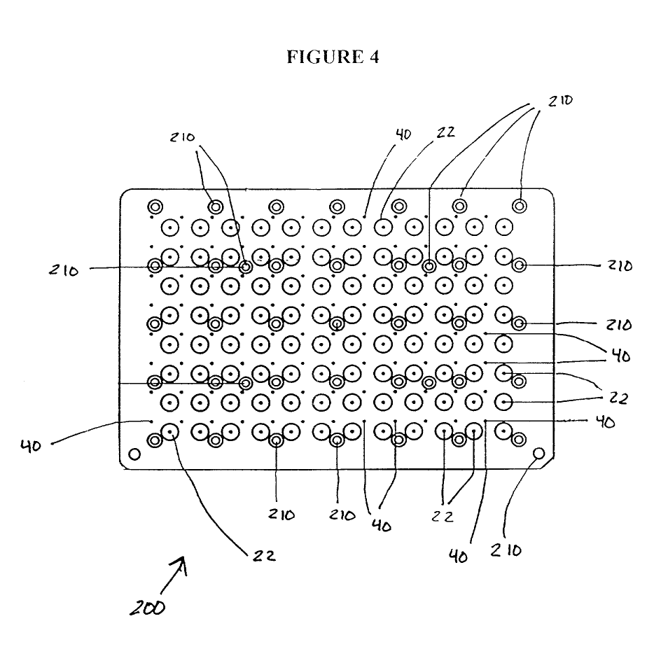

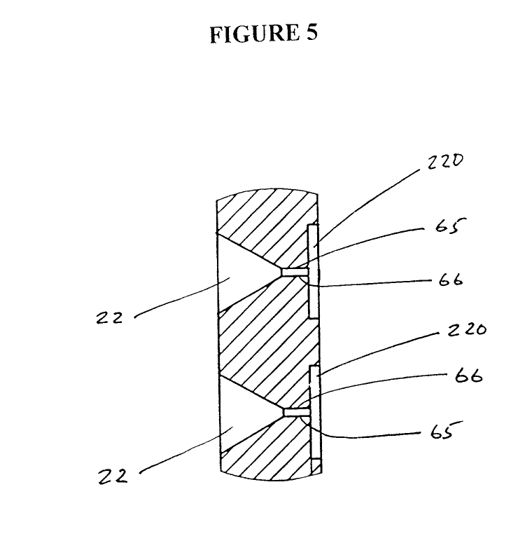

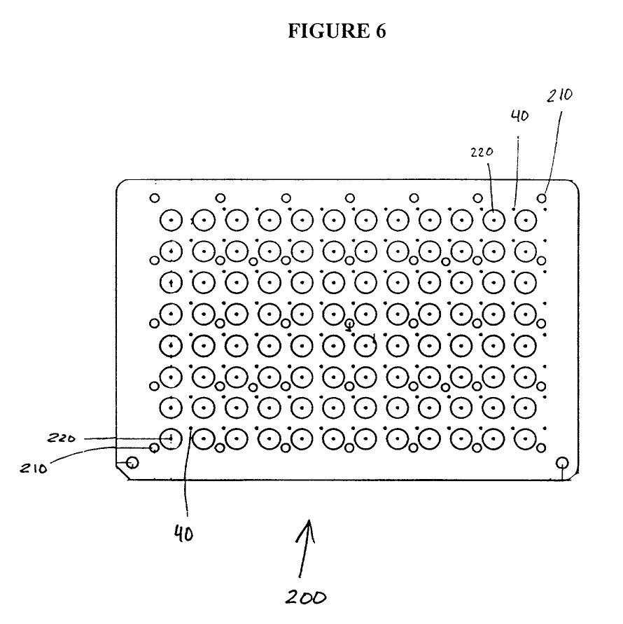

A cross-section of the needle guide 200 shown in FIG. 4 is provided in FIG. 5. Entry portions 22 are fluidly coupled via an upper portion 66 of the guide channel 65, preferably to an annular seal receiving portion 220. As shown in FIG. 6, the bottom surface of needle guide 200 preferably provides an annular seal receiving portion 220 for each flat plate dialysis cell 10. FIG. 6 also illustrates a vent hole 40 corresponding to each annular seal receiving portion 220.

As shown in FIG. 3, the optional seal 300 is preferably provided between the needle guide 200 and the upper channel plate 400. The seal 300 is preferably configured so as to mate with the annular seal receiving portion 220 to provide a fluid-tight seal along the guide channel 65.

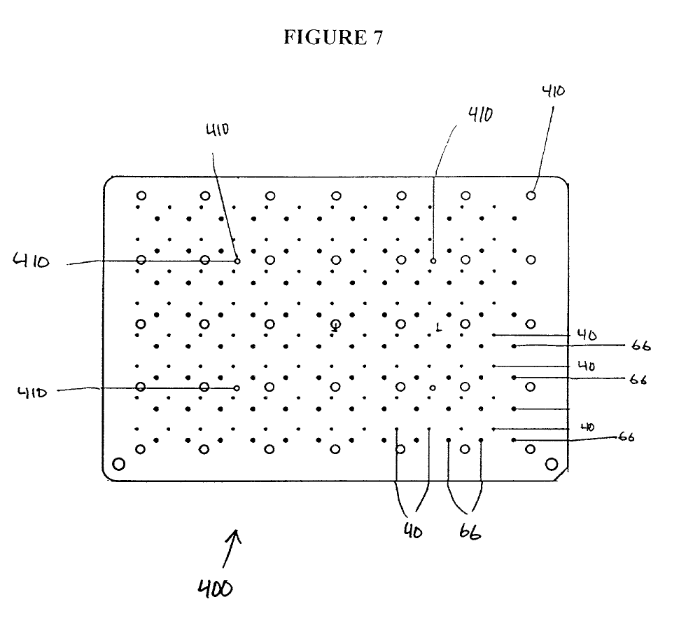

The upper channel plate 400 is described with reference to FIG. 7. FIG. 7 illustrates a pattern of holes similar to those provided in the needle guide 200 in that a pair of two holes is provided for each flat plate dialysis cell 10. However, the upper channel plate 400 differs from the needle guide 200 in that the upper channel plate 400 preferably provides a needle stop, analogous to needle stop 80 of the first embodiment of the invention. An upper portion 66 of the guide channel 65 corresponding to a flat plate dialysis cell 10 is shown in FIG. 7. A corresponding vent hole 40 is also provided, as shown in FIG. 7.

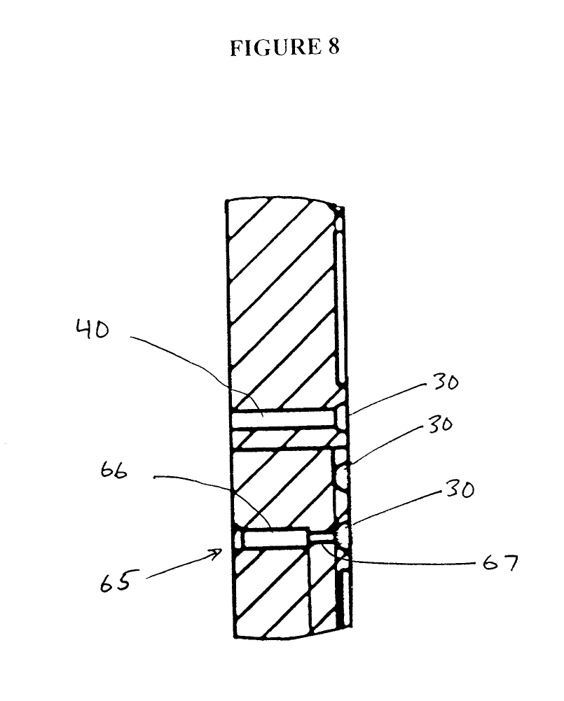

A cross-section of a portion of the upper channel plate 400 is provided in FIG. 8. A guide channel 65 is shown having an upper portion 66 and a lower portion 67. The lower portion 67 of the guide channel 65 preferably has a needle stop formed by a reduced diameter so as to prevent a needle from traveling within the lower portion 67 of the guide channel 65. A vent hole 40 is also provided within the upper channel plate 400. The vent hole 40 may be provided with a varying diameter. FIG. 8 also illustrates a cross-section of the sample chamber 30 in fluid communication with the lower portion 67 of the guide channel 65 and the vent hole 40.

It is within the scope of the scope of the invention to provide an optional seal valve in or in fluid communication with the vent hole 40. Such a seal may be provided to facilitate elevated or reduced pressure within the sample chamber 30.

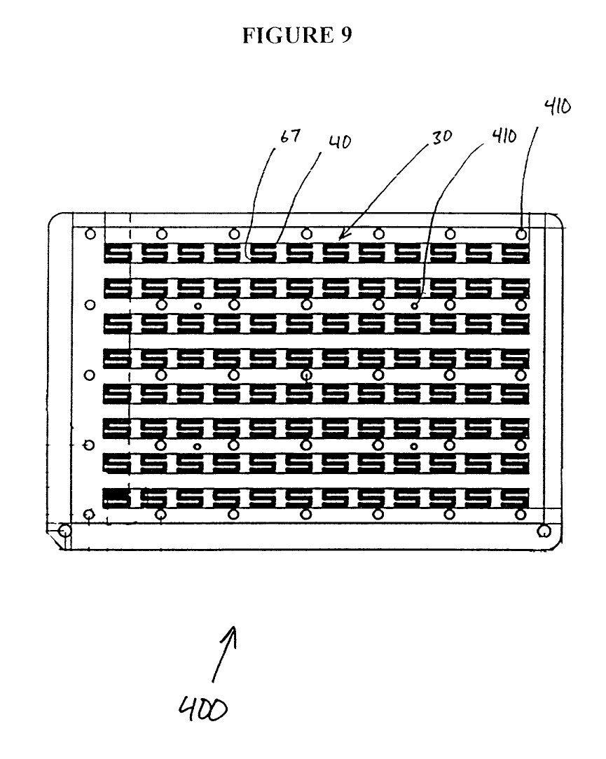

FIG. 9 illustrates a bottom surface of the upper channel plate 400. A sample chamber 30 is provided for each flat plate dialysis cell 10. A vent hole 40 and a lower portion 67 of the guide channel 65 are illustrated in FIG. 9 and correspond to those shown in FIG. 7. Alignment holes 410 are preferably provided within the upper channel plate 400 to correspond to the alignment holes 210 of the needle guide 200.

It is within the scope of the invention to integrally form the needle guide 200 and the upper channel plate 400 in a unitary piece.

As shown in FIG. 3, the membrane 500 is provided between the upper channel plate 400 and the lower channel plate 600. The membrane 500 may optionally be bonded to upper channel plate 400 or may be mounted by a compressive force applied to keep the upper channel plate 400 and the lower channel plate 600 together. Bonding may be performed by ultrasonic welding, heat bonding or a variety of adhesives. A wide variety of membranes 500 may be used depending on the desired operation for flat plate dialysis system 100.

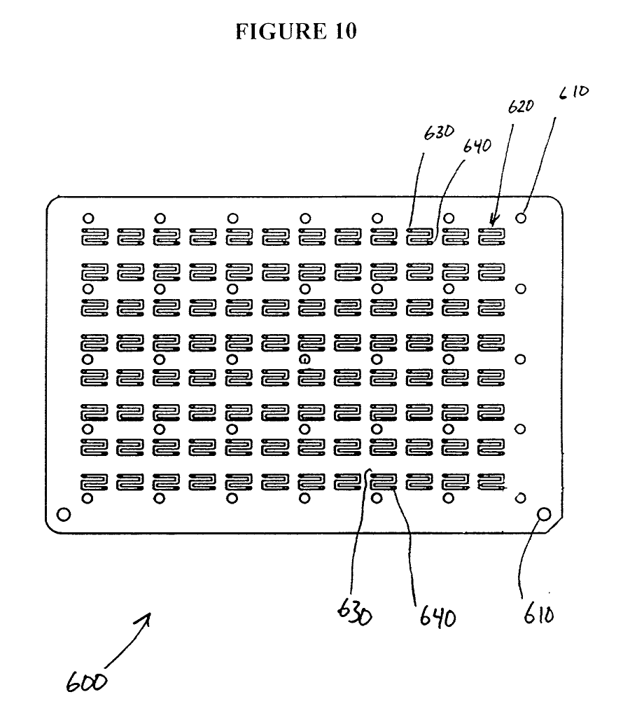

As shown in FIG. 10, optionally, an upper surface of the lower transfer plate 600 provides fluid transfer chambers 620 to correspond to the sample chambers 30 of the upper channel plate 400 shown in FIG. 9. In one variation, the surface areas and volumes of each corresponding sample chamber 30 and fluid transfer chamber 620 are equal, respectively. Equalization of the surface area on each side of the membrane 500 provides an improved structure for equilibrium dialysis by minimizing any pressure differential across the membrane 500. Another variation involves using an inverted upper channel plate 400. Such a variation could also involve a second needle guide 200, resulting in a virtually identical structure on each side of the membrane 500.

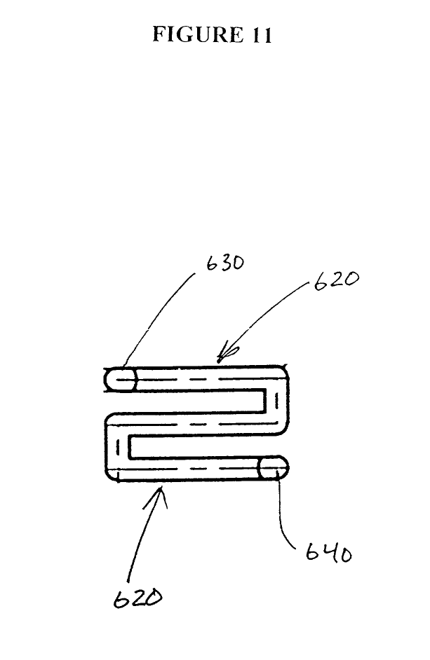

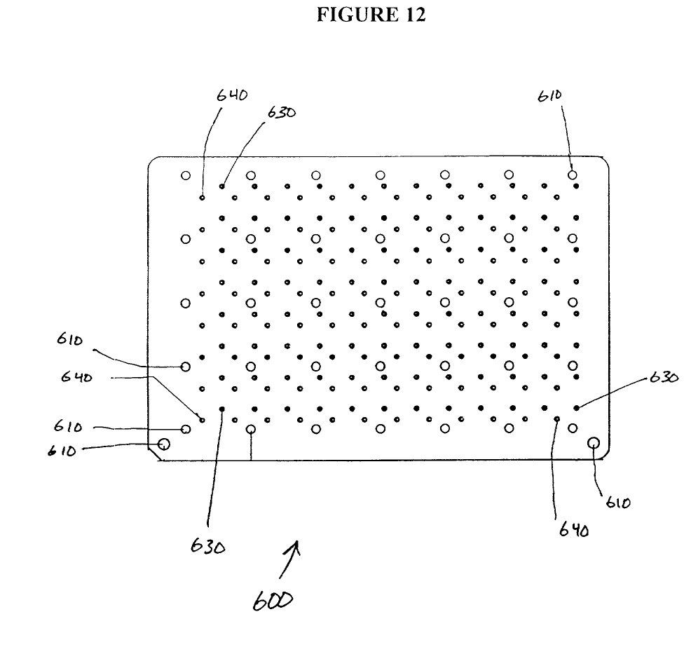

Each fluid transfer chamber 620 is provided with a first port 630 and a second port 640. A detailed view of the fluid transfer chamber 620 is provided in FIG. 11. FIG. 12 shows a bottom surface view of the lower channel plate 600. Alignment holes 610 are optionally provided within the lower channel plate 600 to correspond to the alignment holes in other components of the flat plate dialysis system 100.

It is within the scope of the invention to provide an optional seal or valve within or in fluid communication with the first port 630 and/or second port 640 to aid in altering a pressure within the fluid transfer chamber 620.

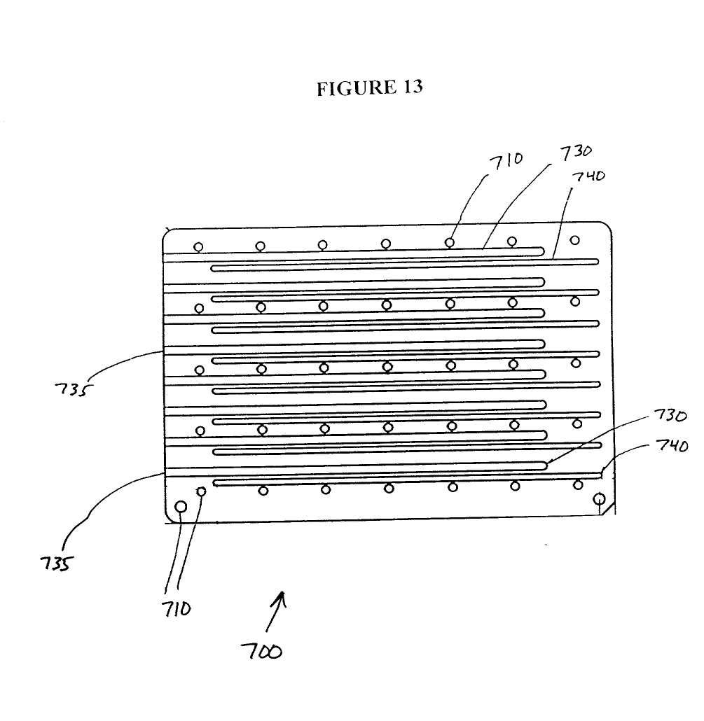

Optionally, a manifold 700 is provided under the lower channel plate 600. Manifold 700, as shown in FIG. 13, provides on an upper surface, a first and a second trough 730, 740. First and second troughs 730, 740, are fluidly coupled to first and second ports 630, 640, respectively, of the lower channel plate 600. First trough 730 fluidly communicates with a first external port 735. Second trough 740 preferably does not communicate with an external port. Both first and second troughs 730, 740 allow fluid communication among first and second ports 630, 640 along a row of flat plate dialysis cells 10 within the flat plate dialysis system 100. Optionally, alignment holes 710 are provided within the manifold 700 to correspond to alignment holes of the other components of the flat plate dialysis system 100.

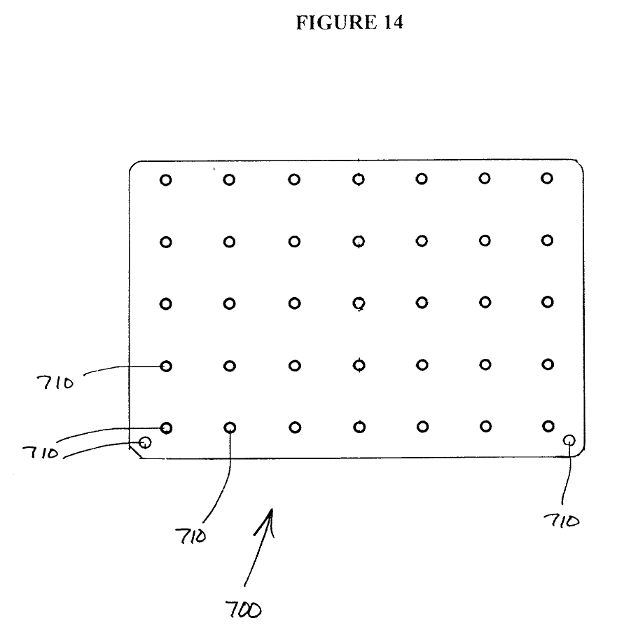

FIG. 14 provides a view of a bottom surface of the manifold 700. The lower channel plate 600 and the manifold 700 are both optional components of the flat plate dialysis system 100. Processing of a sample, such as conducting dialysis or thermocycling, can be performed in the sample chamber 30 by passing a dialysis solution along the membrane 500 with or without the fluid transfer chamber 620 of the lower channel plate 600.

In operation, the flat plate dialysis system 100 is adapted to be used with a syringe needle docking system 95 or a multi-channel pipettor system, such as a 96 or 384 or more channel pipettor. Pipettor syringes are provided to align with the entry portions 22 shown in FIGS. 3, 4 and 5. The needles of the pipettor syringes are inserted into the needle guide 200 each along an insertion axis 90, shown in FIG. 5. The needles travel along the guide channel 65. The guide channel 65 is provided with a larger diameter along an upper portion 66 and a narrower diameter along lower portion 67. The lower portion 67 of the guide channel 65 preferably does not allow the needle to pass within it. A fluid-tight seal is provided by the seal 300 preferably seated within the annular seal receiving portion 220, illustrated in FIGS. 3, 5 and 6.

The needles deposit a sample through the lower portion 67 of the guide channel 65 into the sample chamber 30. As discussed above in relation to the first embodiment of the invention, the sample chamber 30 is preferably formed with a diameter of less than 1 mm and greater than 0.1 mm. A diameter of approximately 0.5 is preferred. The sample chamber is preferably formed in the shape of an elongated tube cut along its longitudinal axis, thereby forming a flat portion along substantially all its length. The sample chamber may be formed in a serpentine shape, such as an S shape, or may be straight. The sample flows freely into the sample chamber 30 due to vent hole 40 allowing the release of air contained within the sample chamber 30. As discussed above, an optional seal 45 or valve may be provided within or in fluid communication with vent hole 40 to regulate the flow through vent hole 40.

Dialysis is conducted by the introduction of dialysis solution into the first trough 730 of the manifold 700. The dialysis solution passes through the first trough 730 into the first port 630, thereby entering the fluid transfer chamber 620. Dialysis solution is introduced sufficiently to allow the dialysis solution to enter the second trough 740 after passing through the fluid chamber 620 and second port 640. A variety of alternative configurations of the trough 700 are within the scope of the invention. For example, first and second troughs 730, 740 may be provided such that a continual flow path is provided. For example, second trough 740 may be fluidly coupled to an external second port to provide for release of dialysis solution introduced into the first external port 735. An increase in flow of the dialysis solution across the membrane 500 typically increases the rate of dialysis. It is within the scope of the invention to integrally form the lower channel plate 600 and manifold 700 in a unitary piece.

Upon completion of the dialysis, preferably, a needle is used to remove the sample from sample chamber 30. The dialysis solution present in the fluid transfer chamber 630 may be removed in advance of, concurrently with or after removal of the sample from sample chamber 30.

The invention is ideally suited for use with equilibrium dialysis, requiring no pressure differential across the membrane 500. However, alternative dialysis and purification methods can be used with the invention.

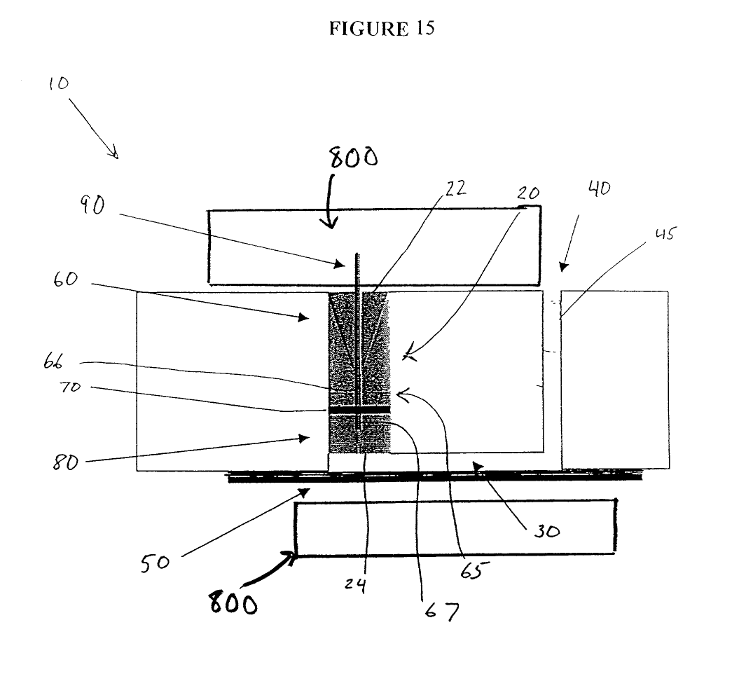

According to an alternate embodiment, the invention may be used to perform ultrafiltration of a sample, as illustrated in FIG. 15. Ultrafiltration involves applying a pressure differential to a sample chamber across the membrane to drive the filtration process. The applied pressure differential may comprise a positive pressure or a negative pressure. The amount of pressure applied in the ultrafiltration process depends upon particular parameters of the flat plate system, the rate of ultrafiltration desired, and the sample being used. For example, the type of membrane being used, the active filtration area of the membrane, the molecular cutoff of the membrane, the strength and thickness of the membrane, the amount of sample to be filtered and the level of polarization of the sample are all factors that affect the amount of pressure used in the ultrafiltration process. Generally, a positive pressure differential between about 0.5 and about 80 pounds per square inch (PSI) may be used, and a negative pressure differential between about 0.5 and about 15 psi may be used to effect filtration of a sample through the membrane.

A pressure plenum 800 for applying a pressure differential to the sample may be utilized to provide ultrafiltration of the sample in the flat plate system of the invention. For example, pressure may be increased within the fluid transfer chamber 630 or the sample chamber 30. To allow the pressure in the sample chamber to be varied, the vent hole 40 of the flat plate system must be sealed using the seal 45 or a valve. For example, to increase the pressure in the sample chamber, the vent is first sealed and additional liquid or gas, such as water or air, can be injected into the sample chamber 30 containing a sample, providing ultrafiltration. Alternatively, or in addition, a pressure plenum 800, providing a positive or negative pressure, may be positioned in fluid communication with the sample chamber to increase or decrease the pressure in the sample chamber, thereby achieving ultrafiltration of the sample. The membrane 50, 500 is preferably configured to prevent DNA from passing through the membrane 50, 500, while allowing impurities to escape. A vacuum may also be applied to either the fluid transfer chamber 620 or sample chamber 30 to provide ultrafiltration, using the device 800 for applying pressure, such as a vacuum manifold or other suitable device. According to one embodiment, a syringe used to inject a sample into the flat plate system may be used to apply a pressure differential to the sample, by expelling a gas or a liquid into the sample chamber 30 containing the sample. As disclosed in relation to the first embodiment of the invention, a variety of processes are within the scope of the invention.

The components of the flat plate dialysis cell 10 and flat plate dialysis system are preferably formed of non-reactive plastic. Specifically, components such as the needle guide 200, upper channel plate 400, lower channel plate 600 and manifold 700 may preferably be formed of hydrophobic materials, such as polystyrene, polycarbonate, TEFLON™, or DELRIN™. Optional coatings of TEFLON™ or silane may also be used to enhance hydrophobic properties of these materials. The membrane 50, 500, for use in dialysis, may preferably be formed of cellulose, cellulose ester, TEFLON™, polysulfone and polyethersulfone. As discussed above, alternative membranes may be used in place of the membrane 50, 500, such as MYLAR™ for thermocycling.

A further variation of the invention involves the use of transparent components of the flat plate dialysis system 100 to allow fluoroscopy. The sample and/or the dialysate may be fluorometrically analyzed. A further variation of the invention allows the use of alignment holes 210, 410, 610 and 710 for the passage of a temperature-controlled solution so as to vary the temperature of the sample chamber 30 and/or fluid transfer chamber 620. Thermocycling may be achieved, for example, by blowing air of different temperatures, although a liquid medium could also be used for heat transfer. Alternatively, additional holes or passages may be provided to allow for the distribution of a temperature controlled fluid to effect the temperature of sample chamber 30 or fluid transfer chamber 620 and the membrane 500.

An additional application of the invention involves using the flat plate dialysis system to detect the presence or absence of a particular nucleotide sequence in a strand of DNA. For example, the flat plate dialysis system can be used with a SNP (single nucleotide polymorphism) assay to detect the presence of a SNP in a strand of DNA. The flat plate dialysis cell 10 may be utilized in conjunction with a SNP assay as described in U.S. Pat. No. 5,391,480, U.S. Pat. No. 5,888,819, U.S. Pat. No. 6,004,744 and U.S. application Ser. No. 60/266,035, the contents of which are herein incorporated by reference, or any suitable technique for detecting the presence or absence of a particular nucleotide within a DNA molecule. Briefly, the SNP assay described in U.S. application Ser. No. 60/266,035 provides a method for detecting the presence or absence of a first nucleotide, at a position within a DNA molecule in a sample by forming an admixture of a primer and a strand of DNA of the sample and imposing conditions such that a hybridization product is formed between the primer and the DNA strand. The primer comprises a sequence of DNA which hybridizes with the strand of DNA adjacent to the first nucleotide position and has a second nucleotide opposite the first nucleotide position. The second nucleotide has an associated label (e.g., a fluorescent label, a radioactive label or a mass-tag) and hybridizes to the first nucleotide in the event that the second nucleotide is complementary to the first nucleotide. The second nucleotide does not hybridize to the first nucleotide in the event that the second nucleotide is not complementary. A proofreading polymerase is applied to the hybridization product under conditions in which the second nucleotide is preferentially excised to form a labeled nucleotide product in the event that the second nucleotide is not hybridized to the first nucleotide, and in which the second nucleotide is preferentially incorporated into a primer extension product in the event that the second nucleotide is hybridized to the first nucleotide.

The presence or absence of a label in excised nucleotides and extension products may then be detected using the dialysis system of the illustrative embodiment. The admixture is injected, e.g., from a needle, through the syringe docking port 30, into the sample chamber 30 of a flat plate dialysis cell 10 and into contact with a first side of the membrane 50. The membrane 50 of the flat plate dialysis cell 10 is selected to have a molecular weight cut-off such that the labeled nucleotide excision product may pass through (or passes through quickly), the primer may not pass through (or passes through slowly), and the extension product may not pass through. A dialysis solution is applied to a second side of the membrane 50 opposite the first side of the membrane to effect separation of the components. The dialysate from the flat plate dialysis cell 10 may then be collected and the presence of the label in the dialysate may be determined using any suitable detection means, e.g., direct fluorescence measurement, or mass spectrometry. The sample on the first side may also be monitored for the presence of a label after providing sufficient time for dialysis of the various components of the sample to occur. The presence of a label in the dialysate in concentrations greater than a background amount after a first predetermined time period (nucleotide excision product) is indicative of the absence of the first nucleotide, and the presence of a label remaining in the sample chamber in concentrations greater than a background amount after a second predetermined time period that is greater than the first predetermined time period (extension product) is indicative of the presence of the first nucleotide. Alternatively, ultrafiltration may be used to separate the labeled nucleotide excision product, the primer, and the extension product by applying a pressure differential to the sample chamber to effect separation and subsequently detecting the sample or the filtrate for the presence of a label.

According to the illustrative embodiment, the membrane has a molecular cutoff of 100 kDaltons. According to the illustrative embodiment, the filtrate is monitored at a time period of between about two minutes and about thirty minutes after the process begins. Preferably, the filtrate is detected about fifteen minutes after the separation of the sample begins. At about fifteen minutes, the ratio of fluorescence between a positive background and a negative result is about 1.5, when a fluorescent label is used. The sample may be detected for the presence of a label after between about thirty minutes and about an hour. Preferably, the sample is detected after about forty-five minutes. At about forty-five minutes, the ratio of fluorescence between a positive result and a negative result is about 1.25 for the sample.

Also within the scope of the invention are various devices to hold the needle guide 200, upper channel plate 400, membrane 500, lower channel plate 600 and manifold 700 together. For example, compression bolts may be provided within the alignment holes 210, 410, 610, 710 of the invention to compress the flat plate dialysis system. Screws may also be used in place of or in combination with compression bolts. Other devices, such as C-clamps or large hose clamps may be used to hold the needle guide 200, upper channel plate 400, membrane 50, lower channel plate 600 and manifold 700 together. Any of the above-described items may also be used with a subset of components of the flat plate dialysis system.

Another variation of the invention involves the use of a beveled corner on each of the needle guide 200, the upper channel plate 400, lower channel plate 600 and manifold 700, or any subset thereof, to aid in alignment of these components of the flat plate dialysis system, as shown in FIG. 3.

The present invention can be used with a conventional fluid dispensing unit, such as a Hydra dispenser, manufactured by Robbins Scientific. Those of ordinary skill will also recognize that other fluid dispensing and sample handling units, whether in modular or discrete forms, can be employed to work with the invention.

The present invention provides benefits over a capillary-type dialysis system. By the nature of its construction, the present invention is less expensive, more durable and more easily constructed and cleaned than a dialysis system using capillaries.

According to a preferred practice, the sample chambers 30 preferably have internal volumes that accommodate fluid sizes of less than about 1 microliter. This sample conservation advantage significantly reduces the sample volumes necessary to achieve selected processing of the sample.

Another advantage of employing submicroliter sample chambers 30 is use of minimal amounts of expensive sequencing reagents and relatively small volumes of biological samples in an automated sample handling format. The invention can be used to perform purification procedures on polymerase chain reaction (PCR) products, preparing sequencing ladders, and injecting the sequencing ladders into appropriate microtiter plates, or aspirating the biological products. Further applications include removal of primers, single nucleotides, fluorescent-labels and salts from PCR reactions, SNP assays and sequencing reactions. Studies on the purification of DNA sequencing reactions by capillary dialysis have demonstrated longer read lengths and higher quality scores than are obtained by conventional alcohol precipitation. Several processing regimens which can be accommodated by the invention are described in detail below.

The following example further describes the feasibility of using membrane dialysis on very small volumes as a method for sequencing, and PCR reaction clean-up. Most of the work was accomplished on sequencing reactions prepared using PE Applied Biosystems standard Big Dye Terminator Cycle Sequencing Ready Reaction Kit, part # 4303154, following the standard ¼ X BigDye Terminator Hydra Sequencing Reactions protocol. The results were obtained on an ABI 377 automated DNA sequencer (PE Applied Biosystems Foster City, Calif. or a Megabase 1000 automated DNA sequencer (Molecular Dynamics Sunnyvale, Calif.). The raw data was analyzed by Phred software (Brent Ewing, LaDeana Hillier, Michael C. Wendl and Phil Green Base-Calling of Automated Sequencer Traces Using Phred I. Accuracy Assessment Genome Research 8, pg 175-185; Brent Ewing, Phil Green Base-Calling of Automated Sequencer Traces Using Phred II. Error Probabilities Genome Research 8, pg 186-194).

Two different flat-sheet dialysis membranes were used for sequencing reaction clean-up. One membrane was a 500,000 MWCO PBVK polysulphone-type membrane, the other a PLCMK cellulosic-type membrane of 300,000 MWCO, both manufactured by Millipore Corp. of Bedford, Mass. Standard ¼ X BigDye Terminator Sequencing Reactions were transferred to the dialysis cassette sample chambers using a Hamilton syringe (Hamilton Company, Reno, Nev.). Distilled water was recycled through the dialysate chambers. After a specified period of time the samples were removed from the dialysis cassette and then run on an ABI 377.

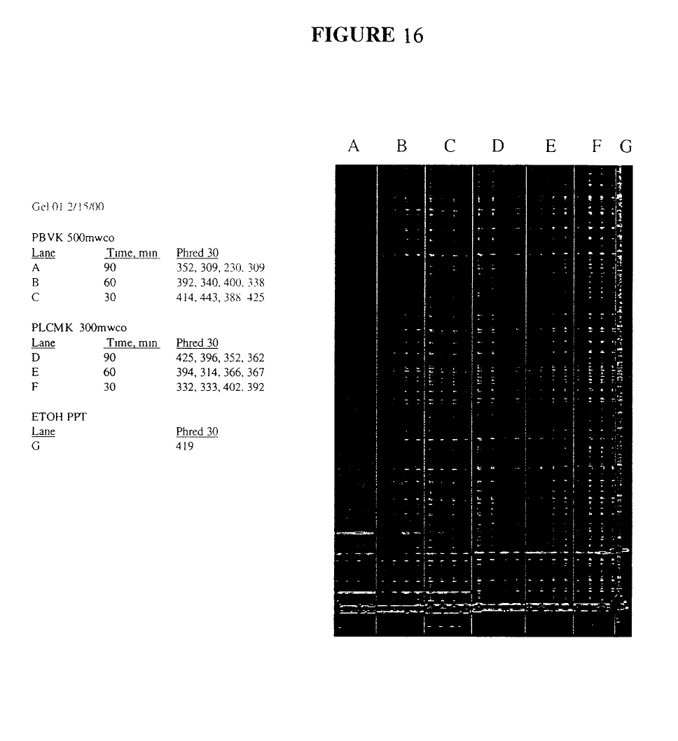

The typical method of removing by-products of the sequencing reaction referred to as “cleaning up” is ethanol precipitation (EtOH PPT.). This protocol is labor intensive and requires the use of a centrifuge. The method of the present invention, which is fast, inexpensive and can be automated, was compared to EtOH PPT. As is evident from FIG. 16, visual inspection of a 377 gel comparing results obtained with the dialysis procedures of the invention versus a standard EtOH PPT. protocol show that the dialysis methodology removes the unincorporated dye terminators which are by-products of sequencing reactions with similar resolution as does EtOH PPT. Also presented are the PHRED quality scores of the sequencing data, for different membranes and different dialysis times versus ETOH PPT. Quality results are also similar in value to ETOH PPT.

According to the invention, purification of a sample may be achieved by a variety of methods, including dialysis, filtration and ultrafiltration. The invention further provides various configurations to achieve purification, depending on the method of purification selected. For example, when equilibrium dialysis is the method of purification, the apparatus of the invention provides at least one sample chamber 30 with a membrane 50 in operative contact with a dialysate, such as, for example, water. A described above, in certain embodiments, the dialysate is contained in fluid transfer chambers 620. When exchange dialysis is the method of purification method, the contents of the fluid transfer chambers 620 may be changed, or the lower channel plate 600 may be removed and optionally replaced by another with fresh dialysate, or by an open bath of dialysate

Typically, DNA sequencing products are purified to remove excess salt, nucleotides and primers from the biological sample. The membrane 50 of the present invention can be employed to purify DNA, excluding the desired products, while concomitantly allowing undesired components to pass therethrough. The DNA sample is cycled through the membrane 50 by pressure optionally formed within the sample chamber 30, thereby resulting in relatively small components being filtered out of the sample.

As set forth herein, the present invention includes dialysis techniques, which may be used effectively to “clean up” polymerase chain reaction (PCR) and cycle sequencing reactions. Until now, one of the problems with conventional dialysis techniques has been one of scale. Typically, dialysis is carried out on relatively large sample volumes of at least 1 mL or more. The typical PCR or sequencing reaction, on the other hand, generally utilizes sample volumes of approximately 10 μL or less, significantly smaller than the sample volumes in conventional dialysis techniques and well suited to the invention.

The invention is usable for processes such as aspirating, mixing, incubating, assaying, cleaning, dialysis, purification, filtering, ultrafiltration, toxicology, thermocycling, such as heating or cooling, performing PCR, detecting the presence or absence of particular nucleotides in a strand of DNA and delivery of the biological sample alone or in a biologically compatible carrier fluid in a selected manner. The invention can be used in place of or in combination with centrifugal separation and/or charge separation.

The present invention is also usable within automated sample handling systems, such as hotel systems, allowing for large-scale automated processing.

The present invention has been described by way of example, and modifications and variations of the exemplary embodiments will suggest themselves to skilled artisans in this field without departing from the spirit of the invention. Features and characteristics of the above-described embodiments may be used in combination. The preferred embodiments are merely illustrative and should not be considered restrictive in any way. The scope of the invention is to be measured by the appended claims, rather than the preceding description, and all variations and equivalents that fall within the range of the claims are intended to be embraced therein.

Having described the invention, what is claimed as new and protected by Letters Patent is:

1. A flat plate dialysis cell, comprising,
a syringe docking port,
a dialysis chamber fluidly coupled to a distal end of said syringe docking port, and
a vent hole fluidly coupled to said dialysis chamber.

2. A flat plate dialysis cell as claimed in claim 1, said syringe docking port further comprising,
a seal capable of providing a fluid-tight seal after being pierced by a needle,
a needle stop capable of preventing a needle from entering said dialysis chamber,
a needle guide formed in a funnel shape in said syringe docking port to guide a needle toward said dialysis chamber.

3. A flat plate dialysis cell as claimed in claim 1, said syringe docking port further comprising a seal capable of providing a fluid-tight seal after being pierced by a needle.

4. A flat plate dialysis cell as claimed in claim 1, said syringe docking port further comprising a needle stop capable of preventing a needle from entering said dialysis chamber.

5. A flat plate dialysis cell as claimed in claim 1, further comprising a needle guide formed in said syringe docking port to guide a needle toward said dialysis chamber.

6. A flat plate dialysis cell as claimed in claim 5, wherein said needle guide is funnel shaped.

7. A flat plate dialysis cell as claimed in claim 1, further comprising a dialysis membrane provided along a portion of said dialysis chamber.

8. A flat plate dialysis cell as claimed in claim 7, wherein said dialysis membrane is a flat sheet.

9. A flat plate dialysis cell as claimed in claim 7, further comprising,
a fluid transfer chamber mated to said dialysis chamber via said dialysis membrane.

10. A flat plate dialysis cell as claimed in claim 7, wherein said dialysis chamber is less than 0.5 mm in height from said dialysis membrane.

11. A flat plate dialysis cell as claimed in claim 7, wherein said dialysis chamber is serpentine.

12. A flat plate dialysis cell as claimed in claim 11, wherein said dialysis chamber is S-shaped.

13. A flat plate dialysis cell as claimed in claim 7, wherein said dialysis chamber is one longitudinal half of an elongated tube having a diameter of between approximately 1.0 mm and 0.1 mm.

14. A flat plate dialysis cell as claimed in claim 7, wherein said dialysis chamber is one longitudinal half of an elongated tube having a diameter of approximately 0.5 mm.

15. A flat plate dialysis cell as claimed in claim 7, wherein said dialysis membrane incorporates reagents suitable for performing a polymerase chain reaction.

16. A flat plate dialysis system, comprising,
a syringe docking port, having a seal capable of providing a fluid-tight seal after being pierced by a needle, a needle stop capable of preventing a needle from entering said dialysis chamber, a needle guide formed in a funnel shape in said syringe docking port to guide a needle toward said dialysis chamber,
an upper channel plate accommodating said syringe docking port, and
at least one dialysis chamber formed along a bottom surface of said upper channel plate.

17. A flat plate dialysis system, comprising,
a needle guide having an upper surface and a bottom surface, said upper surface being adapted to receive a needle,
an upper channel plate mounted along an upper surface to said bottom surface of said needle guide, and
at least one dialysis chamber formed along a bottom surface of said upper channel plate.

18. A flat plate dialysis system as claimed in claim 17, further comprising,
a seal mounted between said needle guide and said upper channel plate.

19. A flat plate dialysis system as claimed in claim 18, wherein said seal is mounted substantially within an annular seal receiving portion formed within said needle guide.

20. A flat plate dialysis system as claimed in claim 17, further comprising,
a dialysis membrane mounted to said bottom surface of said upper channel plate.

21. A flat plate dialysis system as claimed in claim 20, wherein the dialysis membrane incorporates reagents suitable for performing a polymerase chain reaction.

22. A flat plate dialysis system as claimed in claim 17, further comprising,
a plastic barrier mounted to said upper channel plate to allow thermocycling of said dialysis chamber.

23. A flat plate dialysis system as claimed in claim 17, further comprising,
a lower channel plate forming a fluid transfer chamber substantially corresponding to said dialysis chamber, and
a dialysis membrane mounted between said upper channel plate and said lower channel plate.

24. A flat plate dialysis system as claimed in claim 23, wherein said fluid transfer chamber corresponds to said dialysis chamber along said dialysis membrane.

25. A flat plate dialysis system as claimed in claim 17, further comprising, a manifold including,
a first trough having a first port and a first external port, and
a second trough having a second port,
wherein said first and second ports are fluidly coupled to said fluid transfer chamber.

26. A flat plate dialysis system as claimed in claim 25, further comprising,
a second external port fluidly coupled to said second trough.

27. A flat plate dialysis system as claimed in claim 17, further comprising,
a first alignment hole formed in said needle guide, and
a second alignment hole formed in said upper channel plate, configured such that said first alignment hole corresponds to said second alignment hole.

28. A flat plate dialysis system as claimed in claim 27, further comprising,
a compressive device holding said needle guide and said upper channel plate in proximity to each other.

29. A flat plate dialysis system as claimed in claim 16, further comprising,
vent hole having a first diameter in proximity to said dialysis chamber and a second, smaller diameter away from said dialysis chamber.

30. A flat plate dialysis system as claimed in claim 17, further comprising,
a first beveled corner formed on a corner of said needle guide, and
a second beveled corner formed on a corner of said upper channel plate to correspond to said corner of said needle guide having said first beveled corner, to aid in assembly of said flat plate dialysis system.

31. A flat plate dialysis system as claimed in claim 17, wherein said needle guide and said upper channel plate are transparent to facilitate fluoroscopy of a sample in said dialysis chamber.

32. A flat plate dialysis system as claimed in claim 17, further comprising a plastic membrane mounted to said upper channel plate to facilitate thermocycling of a sample in said dialysis chamber.

33. A method for performing dialysis on a biological sample, comprising the steps of,
providing a dialysis chamber having a flat dialysis membrane along one side of said dialysis chamber and having a syringe docking port and a vent hole each located near an opposite end of said dialysis chamber,
injecting a sample from a needle through said syringe docking port into said dialysis chamber and into contact with a first side said dialysis membrane, and
applying a dialysis solution to a second side of said dialysis membrane opposite said first side of said dialysis membrane.

34. A method of dialysis as claimed in claim 33, after said step of applying, further comprising the step of,
removing said sample from said dialysis chamber.

35. A method for performing dialysis on a biological sample, comprising the steps of,
inserting a sample into a serpentine dialysis chamber having a first side of a dialysis membrane along at least one side of said chamber, and
applying a dialysis solution to a second side of said dialysis membrane opposite said first side, to perform dialysis of said sample.

36. A method of dialysis as claimed in claim 35, after said step of applying, further comprising the step of,
removing said sample from said dialysis chamber.

37. A method for conducting dialysis on a biological microsample comprising introducing said microsample into a dialysis chamber having a dialysis membrane for purifying the sample by molecular size discrimination, and allowing said microsample to reside in said dialysis chamber for a time sufficient such that dialysis of said sample is achieved.

38. The method of claim 37, wherein said dialysis is conducted to remove undesired components of a reaction selected from the group consisting of polymerase chain reactions, DNA sequencing reactions, oligonucleotide extension reactions, exonuclease reactions, OLA reactions, hybridization reactions, and allele-specific polymerase chain reactions.

39. The method of claim 37, wherein said microsample comprises a polynucleotide, polypeptide, carbohydrate, or mixtures thereof.

40. The method of claim 39, wherein said polynucleotide comprises DNA.

41. The method of claim 37, wherein said microsample occupies a volume ranging from 10 μl to 0.05 μl.

42. The method of claim 37, wherein said dialysis membrane comprises one or more membrane elements.

43. The method of claim 42, wherein said dialysis membrane comprises a semi-permeable microfiber.

44. The method of claim 43, wherein said dialysis chamber has a molecular weight cut-off about 100 Kdal.

45. A flat plate ultrafiltration system, comprising:
a syringe docking port;
a sample chamber fluidly coupled to a distal end of the syringe docking port; and
an ultrafiltration membrane provided along a portion of the sample chamber.

46. The flat plate ultrafiltration system of claim 45, further comprising a pressure plenum for applying a pressure differential to a sample in the sample chamber.

47. The flat plate ultrafiltration system of claim 45, said syringe docking port further comprising,
a seal capable of providing a fluid-tight seal after being pierced by a needle.
a needle stop capable of preventing a needle from entering said sample chamber.
a needle guide formed in a funnel shape in said syringe docking port to guide a needle toward said sample chamber.

48. A flat plate ultrafiltration system as claimed in claim 45, said syringe docking port further comprising a seal capable of providing a fluid-tight seal after being pierced by a needle.

49. A flat plate ultrafiltration system as claimed in claim 45, said syringe docking port further comprising a needle stop capable of preventing a needle from entering said sample chamber.

50. A flat plate ultrafiltration system as claimed in claim 45, further comprising a needle guide formed in said syringe docking port to guide a needle toward said sample chamber.

51. A flat plate ultrafiltration system as claimed in claim 50, wherein said needle guide is funnel shaped.

52. A flat plate ultrafiltration system as claimed in claim 45, wherein said ultrafiltration membrane is a flat sheet.

53. A flat plate ultrafiltration system as claimed in claim 45, further comprising,
a fluid transfer chamber mated to said sample chamber via said ultrafiltration membrane.

54. A flat plate ultrafiltration system as claimed in claim 45, wherein said sample chamber is less than 0.5 mm in height from said ultrafiltration membrane.

55. A flat plate ultrafiltration system as claimed in claim 45, wherein said sample chamber is serpentine.

56. The flat plate ultrafiltration system as claimed in claim 46, wherein the pressure plenum for applying a pressure differential comprises a syringe.

57. The flat plate ultrafiltration system of claim 56, wherein the syringe is also used to introduce a sample into the sample chamber.

58. A method for performing ultrafiltration on a biological sample, comprising the steps of,
providing a sample chamber having an ultrafiltration membrane along one side of said sample chamber and having a syringe docking port in fluid communication with said sample chamber,
injecting a sample from a needle through said syringe docking port into said sample chamber and into contact with a first side said ultrafiltration membrane, and
applying a pressure differential to the sample chamber to perform ultrafiltration of the sample.

59. The method of claim 58, further comprising the step of removing the sample from the sample chamber.

60. The method of claim 58, wherein the ultrafiltration membrane is a flat sheet.

61. A method for detecting the presence or absence of a first nucleotide, at a position within a strand of DNA in a sample, comprising:
providing a dialysis chamber having a dialysis membrane along one side of said dialysis chamber and having a syringe docking port and a vent hole in fluid communication with said dialysis chamber, said dialysis membrane having a molecular weight cut-off such that a labeled nucleotide excision product passes through the membrane; and
injecting an admixture from a needle through said syringe docking port into said dialysis chamber and into contact with a first side of said dialysis membrane, said admixture comprising a hybridization product formed of a primer and said strand of DNA in said sample, wherein the primer comprises a sequence of DNA which hybridizes with said strand of DNA adjacent to said first nucleotide position and having a second nucleotide opposite said first nucleotide position, said second nucleotide associated with a label, said second nucleotide hybridizing to said first nucleotide in the event said second nucleotide is complementary to said first nucleotide and said second nucleotide not hybridizing to said first nucleotide in the event said second nucleotide is not complementary, and wherein a proofreading polymerase has been applied to the hybridization product under conditions in which said second nucleotide is preferentially excised to form a labeled nucleotide excision product in the event said second nucleotide is not hybridized to said first nucleotide, and in which said second nucleotide is preferentially incorporated into an extension product in the event said second nucleotide is hybridized to said first nucleotide; and
applying a dialysis solution to a second side of said dialysis membrane opposite said first side of said dialysis membrane, to pass a labeled nucleotide excision product through the membrane.

62. The method of claim 61, further comprising the step of monitoring at least one of the group of: the sample on the first side of the dialysis membrane and the dialysis solution on the second side of the dialysis membrane, for the presence of a label, wherein the presence of a label in the dialysis solution in concentrations greater than a background amount after a first predetermined time period is indicative of the absence of the first nucleotide, and the presence of a label remaining in the dialysis chamber in concentrations greater than a background amount after a second predetermined time period greater than said first predetermined time period is indicative of the presence of the first nucleotide.

63. The method of claim 62, wherein the step of monitoring comprises monitoring both the sample on the first side of the membrane and the dialysis solution on the second side of the dialysis membrane.

64. The method of claim 61, wherein the dialysis membrane comprises a flat sheet.

65. A method for detecting the presence or absence of a first nucleotide, at a position within a strand of DNA in a sample, comprising:
providing an ultrafiltration chamber having an ultrafiltration membrane along one side of said ultrafiltration chamber and having a syringe docking port in fluid communication with said ultrafiltration chamber, said ultrafiltration membrane having a molecular weight cut-off such that a labeled nucleotide excision product passes through the membrane; and
injecting an admixture from a needle through said syringe docking port into said ultrafiltration chamber and into contact with a first side of said ultrafiltration membrane, said admixture comprising a hybridization product formed of a primer and said strand of DNA in said sample, wherein the primer comprises a sequence of DNA which hybridizes with said strand of DNA adjacent to said first nucleotide position and having a second nucleotide opposite said first nucleotide position, said second nucleotide associated with a label, said second nucleotide hybridizing to said first nucleotide in the event said second nucleotide is complementary to said first nucleotide and said second nucleotide not hybridizing to said first nucleotide in the event said second nucleotide is not complementary, and wherein a proofreading polymerase has been applied to the hybridization product under conditions in which said second nucleotide is preferentially excised to form a labeled nucleotide excision product in the event said second nucleotide is not hybridized to said first nucleotide, and in which said second nucleotide is preferentially incorporated into an extension product in the event said second nucleotide is hybridized to said first nucleotide; and
applying a pressure differential to the ultrafiltration chamber to pass a labeled nucleotide excision product through the membrane.

66. The method of claim 65, further comprising the step of monitoring at least one of the group of: the sample on the first side of the ultrafiltration membrane and a filtrate on the second side of the ultrafiltration membrane, for the presence of a label, wherein the presence of a label in the filtrate in concentrations greater than a background amount after a first predetermined time period is indicative of the absence of the first nucleotide, and the presence of a label remaining in the ultrafiltration chamber in concentrations greater than a background amount after a second predetermined time period greater than said first predetermined time period is indicative of the presence of the first nucleotide.

67. The method of claim 66, wherein the step of monitoring comprises monitoring both the sample on the first side of the membrane and the filtrate on the second side of the ultrafiltration membrane.

68. The method of claim 65, wherein the membrane comprises a flat sheet.

69. A method of performing a polymerase chain reaction, comprising:
providing a sample chamber having a membrane along one side of said sample chamber and having a syringe docking port in fluid communication with said sample chamber, wherein the membrane is impregnated with nucleotide probes for the performance of a polymerase chain reaction and dried,
injecting a biological sample comprising DNA from a needle of a syringe through said syringe docking port into said sample chamber and into contact with a first side of said membrane and said nucleotide probes impregnated on the membrane; and
imposing conditions on the sample chamber such that a polymerase chain reaction occurs.

70. A packaged kit for performing a polymerase chain reaction, comprising
a flat plate separation system comprising a sample chamber having a membrane along one side of said sample chamber and having a syringe docking port in fluid communication with the sample chamber, wherein the membrane is impregnated with nucleotide probes and dried, said flat plate separation system packaged with instructions for performing a polymerase chain reaction.

71. The packaged kit of claim 70, further comprising a water-impermeable membrane covering the membrane impregnated with nucleotide probes, to seal the sample chamber.

72. A syringe docking port, comprising,
a needle stop capable of stopping a needle,
a seal mounted against said needle stop and capable of providing a fluid-tight seal after being pierced by said needle, and
a needle guide mounted against said seal to guide said needle through said seal and toward said needle stop.
